(19) United States
(12) Patent Application Publication
(10) Pub. No.: US 2006/0078621 A1
(43) Pub. Date: Apr. 13, 2006

(54) Method of providing customized drug delivery systems

(76) Inventors: Robert Scott Wedinger, Randolph, NJ (US); Dev Kumar Mehra, Furlong, PA (US); Wendy Ivy Wilson, Nottingham, MD (US)

Correspondence Address: William Parks;J. M. Huber Corporation, 907 Revolution Street, Havre de Grace, MD, 21078, US

(21) Appl. No.: 10/964,127

(22) Filed: Oct. 13, 2004

(51) Int. Cl. A61K 9/14

(52) U.S. Cl. 424/489

(57) ABSTRACT

A novel method of correlating the disposition of a specific drug in an individual patient to a controlled and modulated delivery system for optimizing therapeutic response of orally ingested dosage forms is provided. Such a method broadly encompasses a first determination of an individual's metabolic rate in terms of absorption of pharmaceutical materials from within the gastrointestinal tract measured as blood plasma concentration over a specific period of time after ingestion or by other commercially available methods and subsequent determination: 1) predicting a proper pharmaceutical compositions, in terms of amount of active available for absorption by the target patient; and 2) amount of such active pharmaceutical ingredient (API) to be formulated within a drug-delivery device that will take into account the unique metabolic profile of the drug (or drugs) in a specific patient. As a result, the API may be formulated as beads, pellets, minitablets, powders, granules, suspensions, and/or emulsions present within the drug-delivery source. As one potentially preferred embodiment, such beads and/or pellets, which may be coated with different polymers and differing levels of coatings, are selected in response to the initial determination of the patient's metabolic profile in order to ensure the specific targeted patient receives the most efficient dosage of the active drug at a rate unique to that individual.

### FIELD OF THE INVENTION

A novel method of correlating the metabolic profile of a specific drug or combination of drugs in an individual patient to a controlled and modulated delivery system for optimizing therapeutic response of orally ingested dosage forms is provided. Such a method broadly encompasses a first determination of an individual's metabolic rate in terms of absorption of pharmaceutical materials from within the gastrointestinal tract measured as blood plasma concentration over a specific period of time after ingestion or by methods commercially available, as a non-limiting example, from companies such as Genelex Corp of Seattle, Wash., and subsequent determination of: 1) predicting a proper pharmaceutical composition, in terms of amount of active available for absorption by the target patient; and 2) amount of such active pharmaceutical ingredient (API) to be formulated within a drug-delivery device that will take into account the unique metabolic profile of the drug (or drugs) in a specific patient. As a result, the API may be preformulated as beads, pellets, minitablets, powders, granules, suspensions, and/or emulsions present within the drug-delivery source. As one potentially preferred embodiment, such beads and/or pellets, which may be uncoated or coated with different polymers and differing levels of coatings, are selected in response to the initial determination of the patient's metabolic profile in order to manufacture a customized final formulation to ensure the specific targeted patient receives the most efficient dosage of the active drug at a rate unique to that individual.

### BACKGROUND OF THE INVENTION AND PRIOR ART

A significant problem has arisen over the years concerning the administration of proper dosages of pharmaceuticals to targeted patients to ensure maximum efficacy of the medicine or medicines prescribed and, simultaneously, to minimize undesired side effects. Most pharmaceuticals are manufactured in a campaign form with a limited range of dosage strengths. For instance, many analgesics are provided by the manufacturer in two different dosage levels (such as 50 mg or 100 mg tablets). A physician or his patient thus has been forced to rely upon a number of different, less than reliable, manners of administering or prescribing a proper dose for maximum effectiveness. As a result, instances have occurred where patients have suffered toxic or adverse reactions due to an overdose of certain pharmaceuticals as well as many examples of ineffectiveness of certain drugs due to the inability of the target patient to absorb sufficient amounts of drugs for salutary treatment to occur. These problems appear to be directly associated with differential metabolic rates and/or genetic profiles varying across the patient population.

It has been known that genetic constitution and other potential factors, such as environmental exposures, and the like, can contribute significantly to the ability of an individual to metabolize different medicines. This phenomenon is pronounced given that the pharmaceutical industry provides uniform drug delivery systems through standard universal doses, to which some patients appear to respond satisfactorily, whereas a great number of other patients either show no response at all, or worse, toxicity to such dosages that have been known to cause fatalities.

In response to such a problem, and other like problems, there have been attempts to facilitate estimating dosing regimens for different patients, as well as the development of controlled release formulations by pharmaceutical manufacturers to produce a standard delivery system that provides a patient with a reliable dosage level. One such development has been to prepare coated particles of pharmaceuticals in order to permit delayed released until the active reaches the small intestine, primarily the duodenum. However, proposed universal dosing modifications to such an extent have met with certain problems and drawbacks as well. For instance, rapid metabolism can lead to absorbed amounts which result in less than the minimum effective concentration (MEC), resulting in marginal bioresponse to the drug. Slow metabolism can lead to drug accumulation in the body which may result in blood levels above the maximum safe level of the therapeutic window resulting in adverse drug reactions. Basically, the general population can be subdivided into three categories based on their genetic predisposition to metabolism of specific drugs. For any given drug, one section of the population metabolizes pharmaceuticals in a manner which allows them to achieve blood levels within the therapeutic window (population B). In other segments, unique metabolism of the drug occurs in slow metabolizers (population C) and fast metabolizers (population A). Due to the existence of such discrepancies, based upon genetic predisposition, there is a definite problem with properly prescribing not only the correct drugs to target patients, but also correct dosages of such pharmaceutical materials.

Likewise, it is generally accepted that each person has his own genetic profile that governs many aspects of his life, including unique drug disposition and metabolism. In a way, such a situation is analogous to mass-production of clothing and/or footwear; very few people exhibit the same characteristics to wear the same size clothing and shoes. Hence, the clothing and footwear manufacturers do not produce the same size and shape in a “one size fits all” approach. To the contrary, such manufacturers generally produce multiple units of various sizes for all different types of people in terms of size and shape. The pharmaceutical industry, however, mass produces the same dosage strength drugs with minimal differences (i.e., some analgesics may be formulated in 50 mg or 100 mg tablets as noted above; there is generally no wide spectrum of such products offered commercially within those two dosing levels). Thus, the ability to consider such differences may also be taken into account subsequent to proper analysis of an individual from a genetic perspective in much the same manner as within other mass-production industries. In fact, there has been a move in the recent past toward individualized patient-care efforts and personalized medicine regimens wherein tailoring of medical procedures and pharmaceutical medicines to a specific person's genetic characteristics is the goal, such as noted within U.S. Pat. No. 6,510,430 to Oberwager et al., and within Norton, Ronald M., “Pharmacogenomics and Individualized Drug Therapy”, Pharmacogenomics, medscapre Pharmacotherapy, 2001.

Numerous techniques exist in the prior art for preparing sustained or controlled release pharmaceutical formulations in attempts to overcome this problem, including, without limitation, surrounding an osmotically active drug core with a semi permeable membrane. In such a manner, the pharmaceutical active becomes released from the drug core over time through exposure to gastrointestinal fluids which permeate the coating membrane and dissolve the active, thus permitting diffusion of the API through the membrane or orifice. Another non-limiting example is the encapsulation of a plurality of beads, pellets, or tablets that are coated with varying levels of diffusion barriers. Upon exposure to gastric fluids, the active may be released via a host of mechanisms such as diffusion, rupturing, eroding, and the like. Yet another manner of providing controlled release pharmaceuticals involves film coating, wherein one of a plurality of films requires drug diffusion through the film or dissolution of the film prior to API release within the body. Yet another manner of providing controlled release pharmaceuticals involves the formulation and compression of erodible or non-erodible, hydrophilic hydrogels or hydrophobic swelling or non-swelling matrices.

Unfortunately, none of these past procedures address the issue of differing metabolic and/or genetic profiles across a range of different target patients. At the very least, such past attempts did not consider the metabolism of specific actives in a single target patient or given target patient population segment (A, B or C) when determining the dose and frequency of the specific API; they all considered each target patient to be the same from a metabolic/genetic profile perspective.

Furthermore, certain sustained release tablet forms are described in U.S. Pat. Nos. 5,427,798, 4,687,660, and Reissue No. 33,994, among many others. Standard formulations include a water insoluble but permeable film coating surrounding the core tablet and a particulate, water-soluble, pore-forming material dispersed within the film coating. Such a system thus provides an osmotic gradient and channel forming system. Typical coatings have included carnauba wax, cysteine hydrochloride, hydroxypropyl methylcellulose, magnesium stearate, microcrystalline cellulose, polyethylene glycol and titanium dioxide. Such sustained release products include uniform dosages of API; however, these tablet forms are not customized to the metabolic rate of any specific target patient, but to a general population.

Another notable issue is lack of patient compliance to a physician-prescribed dosing regimen. At times it is necessary for a physician to recommend greater dosing frequency or change of the dose of the API in order to best ensure drug effectiveness. This requirement of increased compliance has been known to become cumbersome, whether it is a necessity for the patient to split tablets or count numbers of tablets or remember when to take the medication. Simplification of the dosing regimen would thus be a promising step forward for each individual patient to derive maximum benefits from their medication. Again, rate of metabolism of specific drug actives within a given segment of patient population has not been taken into account in conventional drug therapy, particularly as it concerns patient compliance. The correlation between individual metabolic and/or genetic profiles and drug delivery systems is thus an area ripe for investigation, but heretofore unexplored by the pharmaceutical industry to the extent beyond those noted above.

Unfortunately, and as alluded to above, not every patient exhibits the same metabolic and/or genetic profile. Problems still persist with regards to patients with atypical drug absorption capabilities due to differing degrees of metabolism. For instance, a patient with a slow metabolism (slow metabolizers) may experience toxic effects due to insufficient clearance of the drug from the body. In such a situation, the slow metabolic profile of the target patient does not permit “normal” metabolism, therefore necessitating a reduced dose and dosing frequency. Likewise, a patient with a very high metabolic rate (fast metabolizers) will metabolize so rapidly that the MEC threshold may not be achieved, leading to a marginal pharmacological response to the API.

Sects of the population exhibiting such metabolic variability react uniquely to standard drug delivery systems. There has been no attempt made or ability known up to the present to customize tablets and/or capsules to target the continuum of all different types of patients on an individualized metabolic profile basis. Hence, although the ability to provide such customized pharmaceutical delivery systems would provide much safer and potentially effective treatment methods for certain patients, to date no such customization procedure, strategy, or system has been accorded the prescriber by the pharmaceutical and/or over-the-counter drug delivery industry.

### SUMMARY AND OBJECTS OF THE INVENTION

Therefore, it is an object of this invention to provide a more therapeutically beneficial, safe and reliable pharmaceutical delivery system for individual patients through customization of the formulation based on the individual's metabolic and/or genetic profile for a specific active. Another object of the invention is the ability to dose a target patient to achieve optimized absorption of the particular pharmaceutical active or actives delivered in order to provide salutary treatment. Yet another object is to provide an effective pharmaceutical delivery system as above, but also permitting simultaneous administration and eventual effective treatment by a plurality of pharmaceutical actives that may generally exhibit incompatibility when homogeneously blended into a single drug delivery system or may provide synergism via different mechanism.

Accordingly, this invention encompasses a method of providing a pharmaceutical formulation delivered in a form selected from the group consisting of a capsule, a tablet, and any combinations thereof, wherein said method comprises the following sequential steps:

a) initially determining a target patient's individual metabolic and/or genetic profile (Bioavailability and Pharmacokinetics/Pharmacodynamics parameters) for at least one pharmaceutical active;

b) correlating the metabolic and/or genetic profile of step “a” to a required dose of pharmaceutical active needed to provide a sufficient amount of such active for maximum therapeutic effectiveness, thereby;

c) selecting the proper amount of individual pharmaceutical active-containing components selected from the group consisting of preformulated beads, pellets, minitablets, powders, granules, suspensions, emulsions, and any combinations thereof, to meet the correlating determination of step “b” when present within a capsule or tablet; and

d) introducing the amount of individual pharmaceutical active-containing components of step “c” into a customized/individualized capsule or tablet.

Furthermore, this invention encompasses the capsule and/or tablet manufactured by this method. Additionally, this invention encompasses the method of producing a drug delivery system (as defined below) comprised of a plurality of API-containing materials selected from the group consisting of beads, pellets, minitablets, emulsions, suspensions, powders, and any mixtures thereof, wherein said API-containing materials are dispensed into said drug delivery system from a plurality of different bins, wherein each individual bin comprises a uniform dosage and preformulated API-containing materials and each separate bin comprises different dosages and preformulated forms of such API-containing materials, wherein the amount of each dosage and preformulated form of API-containing materials selected for inclusion within said drug delivery system is determined through the correlation of a specific patient's metabolic profile for the API present within said API-containing materials such that the final formulation present within said drug delivery system is customized to the metabolic profile of said specific patient. The final manufactured drug delivery system produced from this method is also encompassed within this invention. For purposes of this invention, the term “preformulation” or “preformulated” is intended to mean any compositions, materials, or the like, manufactured prior to final production of the ultimate drug delivery system.

### DETAILED DESCRIPTION OF THE INVENTION

This invention is based upon the predictive capability of an appropriate drug-delivery system through a single oral dose permitting the correlation of the amount of a pharmaceutical active present within a formulated tablet and/or capsule (hereinafter referred to as “the drug-delivery system”) to the metabolic profile of a specific target patient. In such a manner, the amount of pharmaceutical is delivered in a more targeted fashion such that the metabolism of such an active is accomplished in the most effective therapeutic manner.

The utilization of pharmaceutical treatments is based upon the ability to deliver needed drugs for treatment in a simple, reliable manner. It is thus the aim that such pharmaceutical utilization and delivery within a target patient provides the most effective results for treatment. However, the metabolic rates and/or genetic profiles of individuals have been found to dictate the absorptive capabilities of patients who may be classified as fast, normal or slow metabolizers. For a given patient, if the metabolic rate of a specific drug is too fast, then, without any controlled or modulated drug release, the body will metabolize the API so quickly that the amount absorbed may not reach the threshold for therapeutic effect. On the other hand, too slow a metabolism leads to dangerous spikes in the plasma level concentration of the API upon subsequent dosing, potentially leading to toxic effects therein. It is thus imperative that individualized specific amounts of API be delivered within the circulatory system of a target patient for best treatment.

The present invention provides a manner of mass customization for overcoming these noted deficiencies through the correlation of a patient's specific metabolic and/or genetic profile for any type of pharmaceutical active and the formulation of an individualized capsule and/or tablet form that includes a plurality of different beads, pellets, powders, granules, emulsions, suspensions, and/or minitablets exhibiting different types and levels of coatings thereon and/or inert materials therein to permit tailored dissolution in intended body fluids, thereby permitting release of certain amounts of needed pharmaceutical actives to be absorbed at the correct rate and within the correct region of the gastrointestinal tract for maximum effectiveness of treatment within the target patient's body.

The active substances which can be used according to the invention may be selected without limitation among those belonging to the following groups: analgesic drugs such as, e.g., buprenorphine, codeine, fentanyl, morphine, hydromorphone, and the like; anti-inflammatory drugs such as, e.g., ibuprofen, indomethacin, naproxen, diclofenac, tolfenamic acid, piroxicam, and the like; anthelmintics such as albendazole, flubendazole, ivermectin, diethylcarbamazine citrate and the like. Antibacterials such as aminoglycosides (Kanamycin, Neomycin, and the like), Rifampin, cephalosporins and related beta lactams (Cefazolin, Cefuroxime, Cefaclor and the like), glycopeptides (Vancomycin and the like), penicillins (amoxicillin, ampicillin, carbenecillin, cloxacillin, dicloxacillin, and the like), quinolones (gatifloxcin, ciprofloxacin and the like), sulfonamides (sulfadiazine, sulfamethoxazole, sulfamerazine, trimethoprim, sulfanilamide, and the like), tranquilizers such as, e.g., diazepam, droperiodol, fluspirilene, haloperidol, lorazepam, and the like; cardiac glycosides such as, e.g., digoxin, ouabain, and the like; antiparkinson agents such as, e.g., bromocriptine, biperidin, benzhexol, benztropine, and the like; antidepressants such as, e.g., imipramine, nortriptyline, pritiptylene, lithium carbonate, clozapine, citalopram, fluoxeitine and the like; antineoplastic agents and immunosuppressants such as, e.g., cyclosporin A, fluorouracil, mercaptopurine, methotrexate, mitomycin, and the like; antiviral agents such as, e.g., idoxuridine, acyclovir, vidarabin, and the like; antibiotic agents such as, e.g., clindamycin, erythromycin, fusidic acid, gentamicin, and the like; antifungal agents such as, e.g., miconazole, ketoconazole, clotrimazole, amphotericin B, nystatin, and the like; antimicrobial agents such as, e.g., metronidazole, tetracyclines, and the like; appetite suppressants such as, e.g., fenfluramine, mazindol, phentermin, and the like; antiemetics such as, e.g., metoclopramide, droperidol, haloperidol, promethazine, and the like; antihistamines such as, e.g., chlorpheniramine, chlorpheniramine maleate,terfenadine, triprolidine, and the like; antimigraine agents such as, e.g., dihydroergotamine, ergotamine, pizotyline, and the like; coronary, cerebral or peripheral vasodilators such as, e.g., nifedipine, diltiazem, and the like; antianginals such as, e.g., glyceryl nitrate, isosorbide dinitrate, molsidomine, verapamil, and the like; calcium channel blockers such as, e.g., verapamil, nifedipine, diltiazem, nicardipine, and the like; hormonal agents such as, e.g., estradiol, estron, estriol, polyestradiol, polyestriol, dienestrol, diethylstilbestrol, progesterone, dihyroergosterone, cyproterone, danazol, testosterone, and the like; contraceptive agents such as, e.g., ethinyl estradiol, lynestrenol, etynodiol, norethisterone, mestranol, norgestrel, levonorgestrel, desogestrel, edroxyprogesterone, and the like; antithrombotic agents such as, e.g., warfarin, and the like; diuretics such as, e.g., hydrochlorothiazide, flunarizine, minoxidil, and the like; antihypertensive agents such as, e.g., propanolol, metoprolol such as metoprolol tartrate or metoprolol succinate, clonidine, pindolol, and the like; chemical dependency drugs such as, e.g., nicotine, methadone, and the like; local anesthetics such as, e.g., prilocaine, benzocaine, and the like; corticosteroids such as, e.g., beclomethasone, betamethasone, clobetasol, desonide, desoxymethasone, dexamethasone, diflucortolone, flumethasone, fluocinolone acetonide, fluocinonide, hydrocortisone, ethylprednisolone, triamncinolone acetonide, budesonide, halcinonide, and the like; dermatological agents such as, e.g., nitrofurantoin, dithranol, clioquinol, hydroxyquinoline, isotretionin, methoxsalen, methotrexate, tretionin, trioxsalen, salicylic acid, penicillamine, and the like; vitamins and the like; steroids such as, e.g., estradiol, progesterone, norethindrone, levonorgestrol, ethynodiol, levenorgestrel, norgestimate, gestanin, desogestrel, 3-keton-desogestrel, demegestone, promethoestrol, testosterone, spironolactone, and esters thereof, azole derivatives such as, e.g., imidazoles and mazoles and derivatives thereof, nitro compounds such as, e.g., amyl nitrates, nitroglycerine and isosorbide nitrates, amine compounds such as, e.g., pilocaine, oxyabutyninchloride, benzocaine, nicotine, chlorpheniramine, terfenadine, triprolidine, propanolol, metoprolol and salts thereof, oxicam derivatives such as, e.g., piroxicam, mucopolysaccharides such as, e.g., thiomucasee, opoid compounds such as, e.g., morphine and morphine-like drugs such as buprenorphine, oxymorphone, hydromorphone, levorphanol, hydrocodone, hydrocodone bitratrate, fentanyl and fentany derivatives and analogues, prostaglandins such as, e.g., a member of the PGA, PGB, PGE, or PGF series such as, e.g., misoprostol or enaprostil, a benzamide such as, e.g., metoclopramide, scopolamine, a peptide such as calcitonin, serratiopeptidase, superoxide dismutase (SOD), tryrotropin releasing hormone (TRH), growth hormone releasing hormone (GHRH), and the like, a xanthine such as, e.g., caffeine, theophylline, a catecholamine such as, e.g., ephedrine, salbutamol, terbutaline, a dihydropyridine such as, e.g., nifedipine, a thiazide such as, e.g., hydrochlorotiazide, flunarizine, a sydnonimine such as, e.g., molsidomine, and a sulfated polysaccharide, as well as cholesterol-lowering statin drugs, such as atorvastatin, simvastatin, and the like.

The active substances mentioned above are also listed for illustrative purposes; the invention is applicable to any pharmaceutical formulation regardless of the active substance or substances incorporated therein.

The concentration of the active substance (the dose) within the capsule, tablet, or other delivery system will depend primarily upon the metabolic and/or genetic profile of the individual patient in terms of the specific pharmaceutical active or actives to be delivered, as noted above. A patient's baseline metabolism for a specific drug is determined through any number of ways; however, the easiest and most typical is through blood testing for genetic profiling to confirm upfront that the gene responsible for producing the enzyme that mediates the metabolism of the drug is present in a normal state and not in a polymorphic form. A known dose is given and the blood plasma concentration for the drug is then measured at regular time intervals. A slow metabolic rate would imply the patient's body is very sluggish in metabolizing the drug, resulting in possible drug accumulation leading to toxic levels; a fast metabolizer's body will transform the drug so rapidly that the therapeutic blood levels may not be attained or may be reached for a very short period of time. Normal metabolizers typically exhibit dose-dependent responses to the drug.

The commensurate dose of the API is then calculated based upon the metabolic and/or genetic profile of the patient. This dose is formulated and correlated to a certain amount of pre-formulated and appropriately coated individual beads (as is one non-limiting potentially preferred manner), pellets, and/or properly formulated minitablets, powders, granules, suspensions, and/or emulsions (such as micro emulsions or multiple emulsions) to be included within a capsule and/or tablet for delivery of the pharmaceutical active(s).

As it is apparent from the above, a particulate formulation according to the invention preferably comprises coated beads or pellets or minitablets with the same amount of API and differing amounts of inert materials and differing types and levels of coatings and/or coated beads or pellets or minitablets with the same amount and type of coating with varying amounts of API therein. The coated materials will thus exhibit different drug release profiles, thereby permitting release of the coated active and subsequent exposure to regions of absorption within the target patient's gastrointestinal tract for proper and timely delivery of sufficient amounts of the pharmaceutical active in relation to the needed levels determined via the initial generation of the metabolic profile, as noted previously.

The coating applied on the beads and/or pellets, as well as possibly minitablets, may in principle be any coating such as, e.g., a film coating, a sugar coating, a bioadhesive coating, or a so-called modified release coating. The coating provides a mechanism of obtaining the desired release profile of the active substance included in the cores or, alternatively, masks the taste of bad-tasting active substances, e.g. bitter tasting active substances such as, e.g., noscapine or theophylline. In some cases, the cores according to the invention may contain two or more layers of coating e.g. a first coating which governs the release rate of the active substance and a second layer which is bioadhesive. Other combinations of coatings, including multiple coating configurations, are also within the scope of the present invention.

As mentioned above, the coating may provide the desired properties with respect to release of the active substance, as well as possible taste-masking. Thus, pharmaceutical formulations according to the present invention may be designed to release the active substance immediately upon administration (the materials may be coated or uncoated) or at any suitable time or time period after administration.

A suitable coating for a formulation according to the invention may, for example be a film coating, e.g. a coating based on one or more of the material selected from the following: hydroxypropyl-methylcellulose, ethylcellulose, methylcellulose, hydroxyethylmethylcellulose, hydroxypropylcellulose, carboxymethylcellulose sodium, acrylate polymers (such as, e.g. EUDRAGIT® E, from Rohm Pharma), polyethylene glycols and polyvinylpyrrolidone; a sugar coating; a bioadhesive coating, such as, e.g., a coating comprising a bioadhesive substance such as, e.g. a fatty acid ester such as, e.g., fatty acid esters wherein the fatty acid component of the fatty acid ester is a saturated or unsaturated fatty acid having a total number of carbon atoms of from C₈ to C₂₂; specific examples are glyceryl monooleate, glyceryl monolinoleate, glycerol monolinolenate, or mixtures thereof. Also possible is a modified release coating, such as, e.g., an enteric coating, e.g. a coating which is such that when the coated materials are swallowed, it will be protected from the chemical, enzymatic and other conditions prevailing within the stomach during passage through this part of the digestive system, but will dissolve or otherwise disintegrate within the intestinal tract, thereby releasing the active substance within the intestines. An enteric coating may be based on one or more of the material selected from the following: methacrylic acid copolymers (e.g. EUDRAGIT® L or S), cellulose acetate phthalate, ethylcellulose, hydroxypropylmethylcellulose acetate succinate, polyvinyl acetate phthalate, and shellac; waxes such as, e.g., beeswax, glycowax, castor wax, carnauba wax; hydrogenated oils such as, e.g., hydrogenated castor oil, hydrogenated coconut oil, hydrogenated rape seed oil, hydrogenated soybean oil; fatty acid or fatty alcohol derivatives such as, e.g., stearyl alcohol, glyceryl monostearate, glyceryl distearate, glycerol palmitostearate; acrylic polymers such as, e.g., acrylic resins (EUDRAGIT® RL and RS acrylic resins are copolymers of acrylic and methacrylic acid esters with a low content of quaternary ammonium groups) poly(methyl methacrylate), methacrylate hydrogels, ethylene glycol methacrylate; polylactide derivatives such as, e.g., dl-polylactic acid, polylactic-glycolic acid copolymer; cellulose derivatives, such as, e.g., ethylcellulose, cellulose acetate, cellulose propionate, cellulose butyrate, cellulose valerate, cellulose acetate propionate, cellulose acetate butyrate; vinyl polymers such as, e.g., polyvinyl acetate, polyvinyl formal, polyvinyl butyryl, vinyl chloride-vinyl acetate copolymer, ethylene-vinyl acetate copolymer, vinyl chloride-propylene-vinyl acetate copolymer, polyvinylpyrrolidone; glycols such as, e.g., 1,3-butylene glycol, polyethylene glycols; polyethylene; polyester; polybutadiene; and other high molecular synthetic polymers. Furthermore, the coating compositions for the drug delivery system may be utilized in the manner discussed in detail column 13, line 14-column 14, line 40, within U.S. Pat. No. 5,413,777 to Sheth et al., such passage incorporated herein by reference.

The coating material may be admixed with various excipients such as, e.g., plasticizers; anti-adhesives such as, e.g., silicon dioxide (silica), talc, and magnesium stearate, kaolin; colourants; and solvents in a manner known per se.

Examples of plasticizers for use in accordance with the invention include polyhydric alcohols such as, e.g., propylene glycol, glycerol, and polyethylene glycol; acetate esters such as, e.g., glyceryl triacetate (Triacetin), triethyl acetate, and acetyl triethyl acetate, triethyl citrate; phthalate esters such as, e.g., diethylphthalate; glycerides such as, e.g., acetylated monoglycerides; oils such as, e.g., castor oil, mineral oil, and fractionated coconut oil; and dibutyl sebacate.

In one potential non-limiting embodiment, the coating is applied on the pellets, beads, and/or minitablets from a solution and/or suspension in a non-toxic or low-toxicity organic solvent or in an aqueous medium. The coating may also be applied by electrostatic deposition. Utilization of an aqueous medium is preferred due to safety, economy and environment. The application of the coating, via aqueous and/or organic solvent application, may be performed in a fluidized bed but any suitable coating apparatus may be employed such as those well known by a person skilled in the art (e.g. pan coating, spray-drying, electrostatic coating etc.). When the cores are coated in a fluidized bed apparatus it has proved advantageous to apply the coating composition from a nozzle positioned in the bottom of the fluid bed apparatus, i.e. having the flow of the liquid (the coating composition) and the fluidizing air in a mixed flow except when the coating is performed with a fat or a wax. By using a mixed flow it has been shown that it is possible to coat relatively small particles without agglomeration.

The amount of coating applied on the pellets, beads, and/or minitablets depends, inter alia, on the size of the cores (such as granules, beads or minitablets), the type of coating employed, the amount and type of the active contained in the minitablets and/or beads, and the desired release pattern. In one potentially preferred, but non-limiting, embodiment, a core size of from about 500 to 1400 microns, more preferably from about 600 to about 1200 microns, is utilized with a coating of 0.1-15 % weight gain employed in order to produce thin-coated beads; whereas the same size core (and thus the same amount of active) is supplied, albeit with larger amounts of coatings (such as one set of beads of about 15-25 % weight gain, and a second set of even greater coating amounts, such as from 25-50% weight gain) in order to provide beads that, taken in combination with the first set, exhibit differing dissolution rates. When incorporated together within a capsule (or like delivery source), the differently coated beads will dissolve at different times, thereby providing the target patient with consistent rate of delivery of the API over time commensurate to the metabolic and/or genetic profile of the target patient as previously determined. The specific manner of predicting the desired consistent delivery via this approach is presented below in greater detail with a particular theophylline active (although the approach followed for predicting metabolic rates and relating such to the amount of different specific coated beads, for this non-limiting example, required for customized drug delivery may be utilized for any API). In essence, to achieve the desired drug loading, requisite amounts of minitablets and/or coated beads will be included within the delivery capsule and/or tablet commensurate with the unique metabolic rate and/or genetic profile of the target patient, as discussed above.

Other non-limiting embodiments of the API could include minitablets, wherein the API is either coated on the tablet surface or compacted with a certain amount of inert materials that delay dissolution. Thus, varied formulations of minitablets comprising 1-99 parts of drug mixed with 99-1 parts of appropriate rate controlling excipient included within the drug delivery system will effectuate an analogous result to the coated beads and/or pellets note above. Such excipients can include, without limitation, rate-controlling water-swellable or water-erodible polymers that will react in the gastrointestinal tract to form a gel layer on the minitablet surface through which the API will diffuse/erode over time or will erode over time upon exposure to gastro-intestinal fluids to permit API release. Certain types of such polymers include, again without limitation, hydrocolloids, pectins, alginates, polyacrylamides (and homologues), polyacrylic acids (and homologues), polyethylene glycol, poly(ethylene oxide), polyvinyl alcohol, polyvinylpyrrolidones, starch (and like sugar-based molecules), modified starch, animal-derived gelatin, cellulose ethers (such as carboxymethylcellulose, hydroxyethylcellulose, and the like), and gums, such as carrageenan, guar, agar, arabic, ghatti, karaya, tragacanth, tamarind, locust bean, xanthan, and the like. The amount of excipient present in relation to the API level will determine the rate of API release/diffiusion/erosion over time and can be selected to comport with the metabolic rate of a specific patient.

Other possible non-limiting embodiments of the API are micronized powders produced through but not limited to jet milling and/or powder mixtures produced by methods such as co-grinding via ball milling to facilitate intimate contact between the powders. Introducing differing mixtures of such powdered forms can thus be provided to dissolve in a manner analogous to the coated/uncoated beads and/or pellets noted above as well.

Pharmaceutical actives may also be delivered in the form of granules produced by wet, dry, and/or fluid bed granulation techniques. Modifications of particle aggregates can thus be utilized to provide differing dissolution rates for delayed delivery.

Yet another possible non-limiting embodiment for API delivery is suspending and/or dispersing such powder and/or powder mixtures through ball or colloid milling. Varying the suspending agent viscosity and/or flocculation mechanism can modify the drug release profile as needed. Possible suspending agents include, without limitation, water-soluble polymers, such as certain classes of alkylcelluloses and alkylalkylcelluloses, polyhydric alcohols (such as alkylene polyols and polyalkylene polyols), EO-PO copolymers or block copolymers, and any mixtures or combinations thereof.

Still another potential embodiment of the API includes, again, without limitation, emulsions, such as single, micro-, and multiple emulsions. Combinations of immiscible liquids such as oil and water are admixed with surfactants to form emulsions. The drug may then be dissolved in one of the liquid phases and mixed with the remaining components to form active-containing droplets suspended in solution. Micro emulsions are formulated in the same manner as regular emulsions but yield micelles containing the drug-rich phase and appear transparent to the human eye. Examples of suitable emulsifying agents for this purpose include, without limitation, non-toxic food-grade surfactants, such as alkoxylated alcohols, sulfonated hydrocarbons, silicone-based surface-active agents, and the like.

The active substance contained in the capsule, tablet, or other delivery system may either be present in admixture with the pharmaceutically acceptable inert carrier, or it may be applied on inert cores comprising the pharmaceutically acceptable inert carrier, optionally in admixture with one or more pharmaceutically acceptable excipients (see below). In the latter case, the active substance may be applied by means of methods well known to a person skilled in the art such as, as one non-limiting example, a fluidized bed method. In the prepared materials, the active substance is present in a layer on the outer surface of the uncoated carrier.

Apart from the active substance and the pharmaceutically acceptable inert carrier, the pharmaceutical formulations according to the invention may contain other acceptable pharmaceutical-grade excipients. The pharmaceutically acceptable excipient for use in a particulate formulation according to the invention is generally selected from the group consisting of fillers, binders, disintegrants, glidants, and lubricants; in the following is given a more detailed list of suitable pharmaceutically acceptable excipients for use in formulations according to the invention. The choice of pharmaceutically acceptable excipient(s) in a formulation according to the invention and the optimum concentration thereof cannot generally be predicted and must be determined on the basis of an experimental evaluation of the final formulation. The formulation contains the active substance and the inert carrier in admixture with one or more pharmaceutical grade excipients. These excipients may be, for example, inert diluents or fillers, such as sucrose, sorbitol, sugar, mannitol, microcrystalline cellulose, starches including potato starch, cornstarch, tapioca, rice, and the like, calcium carbonate, sodium chloride, lactose, calcium phosphate, calcium sulfate, or sodium phosphate; granulating and disintegrating agents, for example, cellulose derivatives including sodium carboxymethylcellulose, croscarmellose, starches including sodium starch glycolate, potato starch, cross-linked polyvinylpyrrolidone (such as crospovidone), alginates, or alginic acid; binding agents, for example, sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone such as, e.g, PVP K12, PVP K15, PVP K17, PVP K25, PVP K30, PVP K60, PVP K90, or PVP K120, or combinations thereof, polyvinylacetate, or polyethylene glycol; and lubricating agents including glidants and antiadhesives, for example, magnesium stearate, zinc stearate, stearic acid, silicas, hydrogenated vegetable oils, or talc. Other pharmaceutically acceptable excipients can be colorants, flavoring agents, plasticizers, humectants, buffering agents, etc.

The general amounts of the coating components can be of any level to permit proper dissolution within a target patient's gastrointestinal tract. Likewise, any amount of additives, such as excipients, binders, disintegrating agents, etc., as noted above, may be of any acceptable level, usually from about 0.01 to about 99% by weight of the entire coating and/or minitablet formulation. The amount of active drug present may also be varied within the cores of different coated beads and/or different minitablet formulations present within a single delivery source, if necessary. There is thus no requirement that each bead and/or minitablet utilized within the delivery source (as one non-limiting example, a capsule) remain static for drug content or amount and type of coating applied or for amount and type of matrix polymer content. The important consideration is that the amount of active drug to be metabolized by the target patient is delivered in such a manner that the MEC is at least met over time and that the maximum safe level is not exceeded over the same period. It is this ability to deliver an API that is unique on a customized basis for individual target patients.

One manner of predicting the proper dosing levels of API tailored to an individual's metabolic profile may be accomplished through the utilization of simulations via multiple non-linear regression models or via artificial intelligence. One non-limiting example of use of artificial intelligence in this application is utilizing what is termed artificial neural networks (ANNs) for such a purpose. ANNs are generally known as computer-based programs that attempt to simulate some features of the biological brain such as learning, generalizing or abstracting from experience. Such tools are parallel information processing systems that can develop adaptive responses to environmental information. By feeding certain information and data into an ANN model, it has been found that predictive capabilities of the amount of API as well as the needed delayed dissolution components associated with such API can be made with reliable results. Hence, it has been found surprisingly possible to tailor a dosing regimen to a specific patient's metabolic profile through the utilization of predicting the proper levels of API, etc., in such a manner.

Other examples of artificial intelligence that may be used in this application include expert systems, Bayesian networks, fuzzy logic and knowledge-based software, and all other like forms.

Generally speaking, such non-limiting predictive tools as Artificial Neural Network Development includes a number of components that contribute to the overall results predicted. For example, the software utilized will commonly include non-linear regression model simulation programs, such as, again, without limitation, Alyuda Neurolntelligence version 2.1, from Alyuda Research Inc. In this example, the overall architecture of an ANN instrument will include the following as well:

Feed forward fully connected network

1 input layer with 30 nodes (for multiple measurements)

1 hidden layer

1 output layer with 1 node

a back propagation training algorithm

hidden layer activation function, in this case, a logistic sigmoid function

an output layer error function which utilizes a sum of squares

an output layer activation function, also being a logistic sigmoid function

Modifications to the ANN architecture may include but are not limited to using a feed backward network, using a partially connected network, having multiple input layers with singular and/or multiple nodes, the use of multiple hidden layers with singular and/or multiple nodes, the utilization of multiple output layers with singular and/or multiple nodes, varying activation and/or error functions, varying training algorithms and varying performance limits such as training rate, number of calculation iterations, acceptable network error, and the like.

Into such a program, data may be entered in terms of, for this invention example specifically, the percent of coated and uncoated beads along with coated and uncoated minitablets in formulation from which the rate of release of the API would be determined over time and measured as a percentage of the original API concentration contained within all of the beads and minitablets. The initial data input would first “train” the software to detect the release of API at certain concentrations [with a target point set at a specified error limit (example, network error value≦0.01)] required before the “training” period is deemed successful . From that point, the model developed by the trained ANN would be able to extrapolate predictions and further actual runs would be measured in relation to the predictive results. In applications of ANN such as this, an overall coefficient of determination (R²) value≧0.70 would be considered acceptable and the model's prediction capability to be high. As noted below in the preferred embodiments of this invention, such results are considered useful for the proper determination of dosing regimens and drug delivery system configurations to meet the metabolic profile of a target individual.

In essence, then, one can determine an individual target patient's metabolic profile for a specific drug, input release data for such a drug into an ANN, take the predictive results there from and develop a highly specific quantitative dose and dosing regimen for the patient combining differing levels of controlled dissolution additives within the drug delivery system, dispense controlled dissolution sub units (such as coated and uncoated beads, coated and uncoated minitablets as one example), from a mechanism including multiple labeled bins of such previously formulated, compressed, coated and analytically tested and approved sub units with each bin containing one gauge of coated or uncoated beads, coated or uncoated minitablets, granulation containing labeled amount of drug per gram of granulation, Suspension, Emulsion, Micro-emulsion, Multiple emulsion and the like and having the ability to dispense the determined amount of each type of dosage form from each container into a capsule or tablet. The resultant capsule or tablet would then reflect the very unique dosage strength determined for customized drug delivery and provide means for production of such customized drug delivery systems on an individual basis or population segment (category A, B or C).

Such ANN programs are discussed in greater detail within a number of publications, including, as a general teaching, Hussain, A. S., Yu, X and Johnson, R. D., 1991. Application of Neural Computing in Pharmaceutical Product Development. Pharm. Res., 8, 1248-1252. In terms of attempts at correlating ANN programs to pharmaceutical operations, which do not function in the manner and process followed by the inventive method, one can view Ebube, N. K., McCall, T., Chen, Y. and Meyer, M. C., 1997, Relating Formulation Variables to In Vitro Dissolution Using Artificial Neural Network. Pharm. Dev. Tech., 2, 225-232, Ebube, N. K., Owusu-Ababio, G. and Moji Adeyeye, C., 2000, Preformulation Studies and Characterization of the Physiochemical Properties of Amorphous Polymers Using Artificial Neural Networks, Int. J. Pharm., 196, 27-35, and Kesavan, J. G. and Peck, G. E., 1996, Pharmaceutical Granulation and Tablet Formulation Using Neural Networks, Pharm. Dev. Tech., 1, 391-404 as basic discussions concerning such pharmaceutical applications.

### PREFERRED EMBODIMENTS OT THE INVENTION

The invention is hereinafter more particularly described through the following non-limiting examples. It is noted that specific pharmaceutical actives are utilized within these examples; however, it should be well understood by the ordinarily skilled artisan within the pertinent art that the inventive method may be practiced with any known active. Thus, the specific types listed below are in no way intended to indicate a limitation as to the breadth of this invention.

### EXAMPLES

Several examples of granules, minitablets, and beads were formed and coated with one or more materials providing different release profiles. Theophylline, a commonly prescribed bronchodilator, was used as a representative active ingredient for these examples.

### Example 1

Theophylline granules were manufactured by combining the Theophylline powder with purified water in a 20-quart bowl of a Hobart planetary mixer (Model A-200T) at a speed setting of #1 (approx. 45 rpm). Batch size was 500 g. The amount of water needed was added over 2 minutes. The wet mass was allowed to mix for an additional 2 minutes.

The wet mass was then passed through a model EXDS-60 extruder, (LUWA Corporation, Charlotte, N.C.) in 500 ml-portions at a time. The extruder was operated at 50 rpm and was fitted with a 1.00 mm screen to control the final diameter of the sphere.

The extrudate was then immediately processed in a Spheronizer (Marumerizer, Model Q-230, LUWA Corporation), fitted with a 1 mm scored friction plate, operated at 1000 rpm and having a residence time of 1 minute. The spheronized product was dried on paper lined trays overnight in a hot-air oven at 50° C. The final product was at its equilibrium moisture content. These spheres were then screened to adjust the granule size; retaining the granules between 16 mesh (1180 μM) and 30 mesh (600 μm).

The % Theophylline release of the granules of Example 1 as well as the powder Theophylline starting material determined according to the procedure given in Example 3 are summarized below in Table 2.

### Example 2

Beads of Theophylline and microcrystalline cellulose were formed by mixing equal amounts of anhydrous theophylline and microcrystalline cellulose (AVICEL® 101, FMC Corporation, Philadelphia, Pa.), both previously passed through 20 mesh screen (850 μm), in a twin-shell type blender for 10 minutes. Batch size was 1.0 kg

The blend was collected and charged into a 20-quart bowl of a Hobart planetary mixer and granulated with purified water at a speed setting of #1. The amount of water needed (42.4% w/w) was added over 2 minutes. The wet mass was allowed to mix for an additional 2 minutes.

The wet mass was then passed through a model EXDS-60 extruder, (LUWA Corporation, Charlotte, N.C.) in 600-ml portions at a time. The extruder was operated at 50 rpm and was fitted with a 1.00 mm screen to control the final diameter of the sphere.

The extrudate was then immediately processed in a Spheronizer (MARUMIZER®, Model Q-230, LUWA Corporation), fitted with a 1 mm scored friction plate, operated at 1000 RPM and having a residence time of 1 minute. The spheronized product was dried on paper lined trays overnight in a hot-air oven at 50° C. The final product was at its equilibrium moisture content. These spheres were then screened to adjust the granule size; retaining the beads between 16 mesh (1180 μm) and 30 mesh (600 μm) and then the screened spheres were subjected to various coatings in a fluid bed fitted with a 4″-6″ Wurster insert.

The % Theophylline release from the beads of Example 2 determined according to the procedure given in Example 3 is summarized below in Table 2.

### Example 3

Minitablets were prepared in a standard fashion by mixing 60% by weight of anhydrous theophylline with silicified MCC, RXCIPIENT® FM1000 an engineered calcium silicate from J.M. Huber Corporation, crospovidone, magnesium stearate, and silicon dioxide. The resulting formulation was then compressed on a Riva—Piccola 10 station rotary tablet press to a target weight of five (5) mg per tablet using 1.5 mm tooling The tablets were compressed in the laboratories of SMI Corp, of Lebanon, N.J.

The release of the active drug Theophylline from the granules, beads and minitablets prepared above in Examples 1-3 was determined utilizing a modification of the Test Method 9 of the theophylline extended release capsule monograph (USP 27/NF XXII, United States Pharmacopeia, 2004) wherein the subject active was exposed to two different successive media: first, 900 mL of 0.1 N hydrochloric acid for 2 hours at 37° C. (monograph—1 hour) within a basket which was stirred at 100 rpm (monograph—50 rpm); and a second, for 16 hours within 900 mL of simulated intestinal fluid without enzyme present at 37° C. (0.1 M potassium phosphate buffer solution, pH 6.8)(monograph—5-10 hours). Table 1 reflects guidelines for the percentage of theophylline released over time needed to meet USP standards for dissolution of the active formulated as an extended release capsule in accordance with Test 9 of the monograph.

TABLE 1
Standard Theophylline Release Over Time (USP monograph)

| Time (hour) | Percent Theophylline Released |
|---|---|
| 1 | 5-15% |
| 2 | 25-45% |
| 3 | 50-65% |
| 4 | ≧70% |
| 6 | ≧80% |

A sample of 600 mg of beads was tested using a Distek Evolution 6100, (Distek, Inc., North Brunswick, N.J.) and a OPT-DISS fiber optic UV dissolution tester, model OPT.6CHSYS, (Leap Technologies, Carrboro, N.C.). The UV absorbance was monitored at 271 nm until 100% Theophylline release was achieved or the run was terminated due to the length of the analysis time. The % Theophylline release was correlated to Theophylline concentration versus UV absorbance. The resulting % release values were grouped as “Fast Release” for those having ≧75% release after 2 hr; “Medium Release” for those having ≧75% release after 6 hr; and “Slow Release” for those having ≧75% release after 12 hr. Release results for these examples are given in Table 2. The t_{50%} is time needed for 50% of the mass of the theophylline present initially to be released.

TABLE 2
% Theophylline Released

| Ex | | | | | | | | | Release | |
|---|---|---|---|---|---|---|---|---|---|---|
| No. | Description | 1 hr | 2 hr | 3 hr | 4 hr | 6 hr | 12 hr | 18 hr | Category | t_{50%} |
| 1 | Theophylline | 100 | 100 | 100 | 100 | 100 | 100 | 100 | Fast | <1 min |
| | Powder | | | | | | | | | |
| 2 | Theophylline | 100 | 100 | 100 | 100 | 100 | 100 | 100 | Fast | 3 min |
| | Granules | | | | | | | | | |
| 3 | Theophylline- | 98 | 98 | 98 | 98 | 98 | 98 | 98 | Fast | 5 min |
| | MCC Granules | | | | | | | | | |
| 4 | Theophylline | 95 | 95 | 95 | 95 | 95 | 95 | 95 | Fast | <1 min |
| | Minitablets | | | | | | | | | |

### Examples 4-5

In these examples the screened beads of Example 2 were coated with a layer of one coating material in a fluid bed coater at The Coating Place in Verona, Wisc. Accurately weighed 600 g of the beads of Example 2 were loaded into a fluid bed column preheated to a chosen temperature and fluidized by adjusting the air flow rate, expressed in cubic feet per minute (cfm). The chosen coating material was prepared by mixing the amounts of ingredients given in Table 2 and the coating composition was then pumped through a nozzle located at the bottom of the spray chamber at a chosen rate expressed in gram/minute with atomization accomplished by adjusting the atomization air pressure, expressed in pounds per square inch (psi). Weight of coating material added at time T was recorded during the trials. The coating level (w/w %) was determined by the amount of coating material applied to the beads at a given time. In most trials, samples were collected at 2 different coating weights with the second coating level of a particular trial being designated by “A”. Coating composition and process values are summarized in Table 3.

TABLE 3

Example No.

| | 4 | 4A | 5 | 5A |
|---|---|---|---|---|
| % coating applied | 5 | 10 | 5 | 10 |
| Ex 2 granules, g | 600 | 600 | 600 | 600 |
| Coating Composition | | | | |
| Purified H₂O, g | 47 | 47 | 84 | 84 |
| SURELEASE ®, g | 233 | 233 | 0 | 0 |
| AQUACOAT ® ECD30, g | 0 | 0 | 200 | 200 |
| CITROFLEX ® 2 | 0 | 0 | 14.4 | 14.4 |
| Coating variables | | | | |
| Inlet Air Temp., ° F. | 140 | 140 | 140 | 140 |
| Fluidization air, cfm | 22 | 22 | 23 | 23 |
| pump rate g/min | 5.1 | 5.1 | 6.5 | 6.5 |
| atomization air, psi | 18 | 18 | 16 | 16 |

SURELEASE ® is a 25% aqueous dispersion of ethylcellulose available from Colorcon, West Point, PA;
AQUACOAT ® ECD30 is a 30% aqueous pseudo-latex of ethylcellulose available from FMC Corporation, Philadelphia, PA; and
CITROFLEX ® 2 is triethyl citrate used as a plasticizer and is available from Marflex, Greensboro, NC.

The release of the active drug Theophylline from the coated granules prepared above was determined utilizing the modified USP method for extended release theophylline capsules discussed earlier, using a Distek Evolution 6100, (Distek, Inc., North Brunswick, N.J.) and a OPT-DISS fiber optic UV dissolution tester, model OPT.6CHSYS, (Leap Technologies, Carrboro, N.C.) by placing 600 mg of the granules in 900-ml of 0.1 N HCl for 2 hours at 37° C. After 2 hours, the granules were transferred to 900-ml of 0.1 M potassium phosphate buffer solution (pH 6.8, 37° C.) and the UV absorbance at 271 nm was monitored until 100% Theophylline release was achieved or the run was terminated due to the length of the analysis time. The % Theophylline release was correlated to Theophylline concentration versus UV absorbance. The resulting % release values were grouped as “Fast Release” for those having ≧75% release after 2 hr; “Medium Release” for those having ≧75% release after 6 hr; and “Slow Release” for those having ≧75% release after 12 hr. Release results for these examples are given in Table 4. The t_{50%} is a measure of time needed for 50% of theophylline present initially to be released.

TABLE 4
% Theophylline Released

| | | | | | | | | | Release |
|---|---|---|---|---|---|---|---|---|---|
| Ex No. | Description | 1 r | 2 hr | 3 hr | 4 hr | 6 hr | 12 hr | 18 hr | Category |
| 4 | 5% | 96 | 97 | 98 | 98 | 98 | 98 | 98 | Fast |
| | SURELEASE ® | | | | | | | | |
| 4A | 10% | 81 | 86 | 86 | 89 | 90 | 90 | 91 | Fast |
| | SURELEASE ® | | | | | | | | |
| 5 | 5% | 93 | 93 | 93 | 93 | 93 | 93 | 93 | Fast |
| | AQUACOAT ® | | | | | | | | |
| 5A | 10% | 89 | 92 | 92 | 92 | 92 | 92 | 92 | Fast |
| | AQUACOAT ® | | | | | | | | |

### Examples 6-11

In these examples, the screened theophylline beads of Example 2 were coated with a layer of two coating materials in a fluid bed coater following the procedure given in Example 4-5 above. The chosen coating materials were prepared by mixing the amounts of ingredients given in Table 5 below.

TABLE 5
Coating Composition

| | Example No. | | | | | |
|---|---|---|---|---|---|---|
| | 6 | 7 | 8 | 9 | 10 | 11 |
| Purified water, g | 939 | 440 | 318 | 440 | 726 | 726 |
| METHOCEL ® E5 HPMC, g | 48 | 0 | 0 | 0 | 0 | 0 |
| SUREALEASE ® g | 240 | 0 | 0 | 0 | 0 | 0 |
| CITROFLEX ® 2, g | 0 | 0 | 13 | 0 | 40 | 40 |
| POLYGLOSS ® 90, g | 0 | 60 | 70 | 60 | 60 | 60 |
| EUDRAGIT ® RL30D, g | 0 | 0 | 0 | 0 | 668 | 0 |
| EUDRAGIT ® RS30D, g | 0 | 0 | 0 | 0 | 0 | 668 |
| EUDRAGIT ® L30D, g | 0 | 0 | 249 | 0 | 0 | 0 |
| EUDRAGIT ® NE30D, g | 0 | 0 | 0 | 400 | 0 | 0 |
| EUDRAGIT ® FS30D, g | 0 | 400 | 0 | 0 | 0 | 0 |

METHOCEL ® E5 is hydroxypropylcellulose methylcellulose available from Dow Corporation, Midlands, MI;
SURELEASE ® is a 25% aqueous dispersion of ethylcellulose available form Colorcon, West Point, PA;
CITROFLEX ® 2 plasticizer is triethyl citrate available from Marflex, Greensboro, NC;
POLYGLOSS ® 90 is kaolin available from J. M. Huber, Macon, GA;
all grades of EUDRAGIT ® copolymers are available from Degussa Rohm Pharma Polymers, Piscataway, NJ.

In these trials, samples were collected at 2 different coating weights with the second coating level of a particular trial being designated by “A”. Coating process values are summarized in Table 6.

TABLE 6
Coating Parameters

| Exam- | | % | Tempera- | Fluid- | | atom- |
|---|---|---|---|---|---|---|
| ple | Ex. 2 | coating | ture | ization | pump rate | ization |
| No. | Beads, g | applied | ° F. | air, cfm | g/min | air, psi |
| 6 | 600 | 5 | 170 | 22 | 4.5 | 16 |
| 6A | 600 | 10 | 170 | 22 | 4.4 | 18 |
| 7 | 587 | 10 | 130 | 22 | 6.7 | 16 |
| 7A | 587 | 20 | 130 | 22 | 6.7 | 16 |
| 8 | 600 | 10 | 130 | 22 | 6.8 | 16 |
| 8A | 600 | 20 | 130 | 22 | 6.8 | 16 |
| 9 | 600 | 10 | 100 | 23 | 5.5 | 16 |
| 9A | 600 | 20 | 100 | 23 | 5.5 | 16 |
| 10 | 600 | 10 | 135 | 22 | 6.7 | 16 |
| 10A | 600 | 20 | 135 | 22 | 6.7 | 16 |
| 11 | 600 | 10 | 135 | 22 | 6 | 16 |
| 11A | 600 | 20 | 135 | 22 | 6 | 16 |

The release profiles for these examples were determined as described above under Examples 4-5 and the results are summarized below in Table 7.

TABLE 7
% Theophylline Released

| | | | | | | | | | | |
|---|---|---|---|---|---|---|---|---|---|---|
| 11 | 10% | 5 | 11 | 15 | 19 | 26 | 45 | 60 | Slow | 14 hr |
| | EUDRAGIT ® | | | | | | | | | |
| | RS30D/Kaolin | | | | | | | | | |
| 11A | 20% | 5 | 9 | 12 | 15 | 19 | 33 | 44 | Slow | >18 hr |
| | EUDRAGIT ® | | | | | | | | | |
| | RS30D/Kaolin | | | | | | | | | |

### Examples 12-15

In these examples, the screened beads of Example 2 were coated with a layer of three coating materials in a fluid bed coater following the procedure given in Example 4-5 above. The chosen coating materials were prepared by mixing the amounts of ingredients given in Table 8 below.

TABLE 8

Example No.

| | 12 | 13 | 14 | 15 |
|---|---|---|---|---|
| Purified water, g | 295 | 297 | 302 | 251 |
| Citroflex 2, g | 1.3 | 4 | 6.6 | 11.8 |
| POLYGLOSS ® 90, g | 39 | 36.9 | 34 | 33 |
| EUDRAGIT ® L30D, g | 24.7 | 75.1 | 125 | 224 |
| EUDRAGIT ® FS30D, g | 240 | 187 | 133 | 80 |

In these trials, samples were collected at 2 different coating weights with the second coating level of a particular trial being designated by “A”. Coating process parameters are summarized in Table 9.

TABLE 9
Coating Parameters

| | Example No. | | | | | | | |
|---|---|---|---|---|---|---|---|---|
| | 12 | 12A | 13 | 13A | 14 | 14A | 15 | 15A |
| % Coating | 10 | 20 | 10 | 20 | 10 | 20 | 10 | 20 |
| Applied | | | | | | | | |
| Ex. 2 Beads, g | 600 | 600 | 600 | 600 | 600 | 600 | 600 | 600 |
| Temperature, | 130 | 130 | 130 | 130 | 131 | 131 | 131 | 131 |
| ° F. | | | | | | | | |
| Fluidization | 22 | 22 | 22 | 22 | 22 | 22 | 22 | 22 |
| Air, cfm | | | | | | | | |
| Pump Rate, | 6.8 | 6.8 | 7.2 | 7.2 | 7 | 7 | 6.5 | 6.5 |
| g/min | | | | | | | | |
| Atomization | 16 | 16 | 16 | 16 | 16 | 16 | 16 | 16 |
| Air Pressure, | | | | | | | | |
| psi | | | | | | | | |

The release profile for these examples were determined as described above under Examples 4-5 and the results are summarized below in Table 10.

TABLE 10
% Theophylline Released

| | | | | | | | | | | |
|---|---|---|---|---|---|---|---|---|---|---|
| 15A | 20% | 1 | 1 | 63 | 81 | 85 | 86 | 87 | Me- | 165 min |
| | EUDRAGIT ® | | | | | | | | dium | |
| | L/FS (7:3) and | | | | | | | | | |
| | Kaolin | | | | | | | | | |

### Examples 16-19

In these examples, the spheres prepared above in Examples 4A, 5A, 6 and 6A were further coated with layer of SURTERIC® polyvinyl acetatephthalate based enteric coating (Colorcon, West Point, Pa.) in a fluid bed coater following the procedure given in Example 4-5 above. (Note that Examples 4A and 5A were previous coated with 10% SURELEASE® and 10% AQUACOAT® ECD30, respectively, and Examples 6 and 6A were previously coated with 5% and 10% of a mixture of METHOCEL® E5 and SURELEASE®, respectively.) The SURTERIC® coating materials were prepared by mixing the amounts of water and powdered SURTERIC® given in Table 11 below and then filtering the coating composition through a 60 mesh sieve (250 μm) before use.

TABLE 11

Example No.

| | 16 | 17 | 18 | 19 |
|---|---|---|---|---|
| Purified Water, g | 840 | 840 | 1365 | 1365 |
| SURTERIC ®, g | 160 | 160 | 260 | 260 |

In these trials, samples were collected at 2 different SURTERIC® coating weight gains with the second coating level of a particular trial being designated by “A”. Coating process parameters are summarized in Table 12.

TABLE 12

| | Example No. | | | | | | | |
|---|---|---|---|---|---|---|---|---|
| | 16 | 16A | 17 | 17A | 18 | 18A | 19 | 19A |
| % | 10 | 20 | 10 | 20 | 10 | 20 | 10 | 20 |
| SURTERIC ® | | | | | | | | |
| Coating | | | | | | | | |
| Applied | | | | | | | | |
| Substrate | 4A | 4A | 5A | 5A | 6 | 6 | 6A | 6A |
| Beads, g | | | | | | | | |
| Substrate, g | 580 | 580 | 600 | 600 | 605 | 605 | 600 | 600 |
| Temperature, | 145 | 145 | 145 | 145 | 145 | 145 | 145 | 145 |
| ° F. | | | | | | | | |
| Fluidization | 22 | 22 | 23 | 23 | 22 | 22 | 22 | 22 |
| Air, cfm | | | | | | | | |
| Pump Rate, | 6.6 | 6.6 | 7 | 7 | 6.8 | 6.8 | 6.6 | 6.6 |
| g/min | | | | | | | | |
| Atomization | 18 | 18 | 18 | 18 | 18 | 18 | 18 | 18 |
| Air Pressure, | | | | | | | | |
| psi | | | | | | | | |

The release profile for these examples were determined as described above under Examples 4-5 and the results are summarized below in Table 13.

TABLE 13
% Theophylline Released

| Ex | | | | | | | | | Release | | |
|---|---|---|---|---|---|---|---|---|---|---|---|
| No. | Description | 1 hr | 2 hr | 3 hr | 4 hr | 6 hr | 12 hr | 18 hr | Category | t_{50%} | |
| 16 | 10% | 14 | 20 | 26 | 27 | 29 | 33 | 36 | Slow | >18 | hr |
| | SURTERIC ® on | | | | | | | | | | |
| | 10% | | | | | | | | | | |
| | SURELEASE ® | | | | | | | | | | |
| 16A | 20% | 8 | 13 | 21 | 24 | 27 | 34 | 41 | Slow | >18 | hr |
| | SURTERIC ® on | | | | | | | | | | |
| | 10% | | | | | | | | | | |
| | SURELEASE ® | | | | | | | | | | |
| 17 | 10% | 32 | 73 | 73 | 87 | 87 | 87 | 87 | Medium | 83 | min |
| | SURTERIC ® on | | | | | | | | | | |
| | 10% | | | | | | | | | | |
| | AQUACOAT ® | | | | | | | | | | |
| 17A | 20% | 10 | 18 | 55 | 67 | 77 | 83 | 84 | Medium | 163 | min |
| | SURTERIC ® on | | | | | | | | | | |
| | 10% | | | | | | | | | | |
| | AQUACOAT ® | | | | | | | | | | |
| 18 | 10% | 53 | 64 | 88 | 92 | 94 | 94 | 94 | Medium | 50 | min |
| | SURTERIC ® on | | | | | | | | | | |
| | 5% | | | | | | | | | | |
| | SURELEASE ®/ | | | | | | | | | | |
| | HPMC | | | | | | | | | | |
| 18A | 20% | 11 | 17 | 74 | 86 | 90 | 91 | 91 | Medium | 149 | min |
| | SURTERIC ® on | | | | | | | | | | |
| | 5% | | | | | | | | | | |
| | SURELEASE ®/ | | | | | | | | | | |
| | HPMC | | | | | | | | | | |
| 19 | 10% | 37 | 48 | 79 | 87 | 92 | 92 | 92 | Medium | 124 | min |
| | SURTERIC ® on | | | | | | | | | | |
| | 10% | | | | | | | | | | |
| | SURELEASE ®/ | | | | | | | | | | |
| | HPMC | | | | | | | | | | |
| 19A | 20% | 5 | 20 | 65 | 79 | 85 | 85 | 86 | Medium | 158 | min |
| | SURTERIC ® on | | | | | | | | | | |
| | 10% | | | | | | | | | | |
| | SURELEASE ®/ | | | | | | | | | | |
| | HPMC | | | | | | | | | | |

### Example 20

In this example, various binary or tertiary combinations of the beads (granules or minitablets) prepared in Examples 1-19 were loaded into capsules and the release profile was determined. For each binary combination, 500 milligrams of each material was combined and mixed for 30 seconds using a SPEEDMIXER® model DAC 150 FV-K available from Siemens Corporation, New York, N.Y. For the tertiary mixture, 333 milligrams of each material was combined and mixed as above. From each mixture, 300 mg of beads were hand loaded into size 1 gelatin capsules and evaluated according to modified USP method for extended release theophylline capsules discussed earlier, using a Distek Evolution 6100, (Distek, Inc., North Brunswick, N.J.) and a OPT-DISS Fiber optic UV dissolution tester, model OPT.6CHSYS, (Leap Technologies, Carrboro, N.C.) by placing the capsules in 900 ml of 0.1 N HCl for 2 hours at 37° C. After 2 hours, the capsules were then transferred to 900 ml of 0.1 M pH 6.8 potassium phosphate buffer solution at 37° C. and the UV absorbance at 271 nm was monitored until 100% Theophylline release was achieved or the run was terminated due to the length of the analysis time. The % Theophylline release was correlated to Theophylline concentration versus UV absorbance. The resulting % release values were grouped as “Fast Release” for those having ≧75% release after 2 hr; “Medium Release” for those having ≧75% release after 6 hr; and “Slow Release” for those having ≧75% release after 12 hr. Results are summarized below in Table 14. The t_{50%} is a measure of time needed for 50% of theophylline present initially to be released.

TABLE 14
% Theophylline Released

| | | | | | | | | | | | Release | | |
|---|---|---|---|---|---|---|---|---|---|---|---|---|---|
| Capsule | Bead 1 | Bead 2 | Bead 3 | 1 hr | 2 hr | 3 hr | 4 hr | 6 hr | 12 hr | 18 hr | Category | t_{50%} | |
| A | 4A | 5A | — | 86 | 91 | 92 | 93 | 94 | 95 | 95 | Fast | 13 | min |
| B | 7A | 5A | — | 100 | 100 | 100 | 100 | 100 | 100 | 100 | Fast | 10 | min |
| C | 8 | 19 | — | 17 | 23 | 77 | 89 | 92 | 95 | 96 | Medium | 139 | min |
| D | 12A | 3 | — | 55 | 55 | 77 | 93 | 100 | 100 | 100 | Medium | 26 | min |
| E | 13A | 19A | — | 7 | 13 | 85 | 97 | 100 | 100 | 100 | Medium | 159 | min |
| F | 14A | 2 | — | 49 | 50 | 87 | 95 | 97 | 98 | 98 | Medium | 115 | min |
| G | 7 | 10 | — | 47 | 59 | 62 | 64 | 65 | 77 | 90 | Slow | 68 | min |
| H | 9 | 9A | — | 2 | 3 | 4 | 5 | 6 | 14 | 22 | Slow | >18 | hr |
| I | 11 | 6 | — | 51 | 54 | 56 | 59 | 60 | 71 | 79 | Slow | 39 | min |
| J | 15A | 16A | — | 1 | 3 | 34 | 45 | 47 | 53 | 56 | Slow | 7.5 | hr |
| K | 17A | 11 | 16 | 12 | 20 | 38 | 43 | 50 | 61 | 68 | Slow | 6 | hr |
| L | 4A | 9 | — | 60 | 63 | 64 | 65 | 66 | 72 | 77 | Slow | 20 | min |

### Utilizing the ANN for Predicting Release of API

Dissolution data contained in Tables 2, 4, 7, 10, 13 and 14 were utilized to develop a model that would predict the composition of a capsule or tablet needed to achieve a desired dissolution profile that would allow for the production of customized drug delivery systems tailored for maximum efficacy for the target patient. Initially, a database of dissolution data for theophylline beads and capsules was manipulated into three sects, training data, validation data, and test data, by the ANN software. This manipulation of data is reflected in Table 15. Based on the sort of the data, the software then determined the best ANN architecture to evaluate release models after 2, 6, 12, and 18 hours of exposure (Q2, Q6, Q12 or Q18). A model was developed for each output. For any given output, the model developed used the other outputs as input data (example, the model for Q2 used output data for Q6, Q12 and Q18 in its analysis)

TABLE 15
ANN Data Subsets
Example No/Capsule
Training Subset - 23 formulations, 74% of data, R² = 0.99

| | | | |
|---|---|---|---|
| 3 | 4A | 6 | 6A |
| 8 | 9 | 9A | 11A |
| 12A | 16 | 17A | 18 |
| 18A | 19A | A | D |
| E | F | G | H |
| I | J | K | |

Validation Subset - 4 formulations, 13% of data, R² = 0.63

| | | | |
|---|---|---|---|
| 4 | 8A | C | L |

Test Subset - 4 formulations, 13% of data, R² = 0.87

| | | | |
|---|---|---|---|
| 11 | 12 | 19 | B |

Based on the optimized architecture, ANN was trained, resulting in a mathematical model for the target output with random batches excluded for use in validation. A R²>0.70 indicates that the resulting model was predictive of dissolution behavior. After training, a portion of the excluded batches was used to validate the model through comparison of the model's predicted output (i.e., Q2, Q6 etc.) to the actual observed output data collected during testing. After validation, the remaining excluded batches were used in much the same manner to test the model and provide a second level of validation. The resulting R² values for training, validation and testing are included in Table 15.

The network was externally validated by preparing capsules containing mixtures of beads not included in the initial training data. These formulations are included in Table 16. The composition of these formulations was used as input for the ANN and the program was allowed to predict the dissolution characteristics based on the validated models for 2, 6, 12, and 18 hours. These batches were then tested and the predicted and actual observed results were compared. A percent error≦10%, based on the comparison of predicted to observed is desired. If the percent error criterion is satisfied, the developed model can now be used to predict the dissolution performance of various combinations of coated theophylline beads.

TABLE 16
External Validation Batches

| | | | | | | | | | | | Release | |
|---|---|---|---|---|---|---|---|---|---|---|---|---|
| Capsule | Bead 1 | Bead 2 | Bead 3 | 1 hr | 2 hr | 3 hr | 4 hr | 6 hr | 12 hr | 18 hr | Category | t_{50%} |
| M | 2 | 19A | — | 56 | 57 | 91 | 100 | 100 | 100 | 100 | Medium | 22 min |
| N | 15A | 5A | 17A | 40 | 45 | 78 | 89 | 95 | 98 | 98 | Medium | 128 min |
| O | 8A | 10A | — | 35 | 43 | 83 | 92 | 95 | 95 | 96 | Medium | 126 min |
| P | 3 | 11 | 12A | 42 | 43 | 51 | 58 | 64 | 69 | 72 | Medium | 176 min |
| Q | 4 | 9 | — | 58 | 62 | 64 | 66 | 69 | 75 | 82 | Slow | 29 min |
| R | 16 | 13A | — | 6 | 9 | 46 | 62 | 71 | 73 | 77 | Slow | 191 min |

The percent dissolved results shown in Table 16 were compared to predicted results obtained using the ANN and the percent error, a measure of how predictive the system was, was calculated. The results of this comparison are listed in Table 17. Of the 24 data points compared, 7 of the comparisons failed to meet the criteria for percent error (i.e., ≦10%) with 2 of the outer comparisons failing by less than 1 percentage point.

TABLE 17
External Validation Percent Error

| | Predicted Percent Dissolved | | | | Percent Error | | | |
|---|---|---|---|---|---|---|---|---|
| Capsule | 2 hr | 6 hr | 12 hr | 18 hr | 2 hr | 6 hr | 12 hr | 18 hr |
| M | 54 | 99 | 97 | 93 | −5.3 | −1.0 | −3.0 | −7.0 |
| N | 46 | 97 | 96 | 92 | 2.2 | 2.1 | −2.0 | −6.1 |
| O | 45 | 96 | 95 | 92 | 4.7 | 1.1 | 0.0 | −4.2 |
| P | 19 | 71 | 73 | 85 | −55.8 | 10.9 | 5.8 | 18.1 |
| Q | 21 | 70 | 83 | 87 | −66.1 | 1.4 | 10.7 | 6.1 |
| R | 1 | 96 | 79 | 82 | −88.9 | 38 | 8.2 | 6.5 |

Based on the assessment of the ANN in terms of R² and percent error in this example, it was determined that the predictive capabilities of the ANN system were sufficient to provide customized drug delivery systems utilizing differing amounts of differently coated beads of API (here, theophylline, as one non-limiting possibility).

While certain preferred and alternative embodiments of the invention have been set forth for purposes of disclosing the invention, modifications to the disclosed embodiments may occur to those who are skilled in the art. Accordingly, this specification is intended to cover all embodiments of the invention and modifications therof which do not depart from the spirit and scope of the invention.

What is claimed is:

1. A method of producing a drug delivery system comprised of a plurality of active pharmaceutical ingredient (API)-containing materials selected from the group consisting of beads, pellets, minitablets, emulsions, suspensions, powders, and any mixtures thereof;
said method comprising dispensing said API-containing materials into said drug delivery system from a plurality of different bins;
wherein each individual bin comprises a uniform API-containing material and each separate bin comprises different API-containing materials having different dosages and formulations as those materials present within different bins;
wherein the total amount of each different API-containing material from at least two of the plurality of different bins selected for inclusion within said drug delivery system is determined through the correlation of a specific patient's metabolic profile for the API present within said API-containing materials;
wherein the selected numbers of different materials from the different bins are then introduced within a drug delivery system; and
wherein the final formulation present within said drug delivery system is customized to the metabolic profile of said specific patient.

2. The method of claim 1 wherein said API-containing materials are selected from the group consisting of beads, pellets, minitablets, and any mixtures thereof.

3. A drug delivery system produced by the method of claim 1.

4. A drug delivery system produced by the method of claim 2.

5. The drug delivery system of claim 3 wherein said system is selected from the group consisting of a capsule, a tablet, and mixtures thereof.

6. The drug delivery system of claim 4 wherein said system is selected from the group consisting of a capsule, a tablet, and mixtures thereof.
